(19) United States
(12) Patent Application Publication
(10) Pub. No.: US 2005/0183150 A1
(43) Pub. Date: Aug. 18, 2005

(54) Method for regenerating and transforming St. Augustinegrass from embryogenic callus

(76) Inventors: Rebecca Torisky, Waterloo, NY (US); Della Cobb, Waterloo, NY (US); Lisa Lee, Marysville, OH (US); Joyce Van Eck, Ithaca, NY (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/777,769

(22) Filed: Feb. 13, 2004

(51) Int. Cl. A01H 1/00; C12N 15/82; A01H 5/00

(52) U.S. Cl. 800/278; 800/320

(57) ABSTRACT

Method of initiating, proliferating, and regenerating embryogenic callus from immature inflorescence explants of St. Augustinegrass, and method of transforming and regenerating the embryogenic callus to produce transgenic St. Augustinegrass. The invention also encompasses St. Augustinegrass callus and adult plants produced by the method.

### BACKGROUND OF THE INVENTION

St. Augustinegrass (Stenotaphrum secundatum) is a popular turfgrass widely used in the southern United States, particularly in the gulf coast region and Florida. St. Augustinegrass is native to the Gulf of Mexico, the West Indies and Western Africa. The species flourishes in warm, moist climates and is a prolific growing species that spreads rapidly by creeping stolons. By appearance, St. Augustinegrass is a coarse textured, stoloniferous species, which roots at the nodes (Duble, R. L., Texas Agricultural Extension Service Internet Publication, 1996).

The growth range of the species is restricted to the gulf coast region and Florida due to intolerance to cold climates. Similarly, the species requires frequent and repetitive watering in order to survive arid climates.

St. Augustinegrass is susceptible to several pests and pathogens that are particularly troublesome for the species. Most notable among the St. Augustinegrass pests is the southern lawn chinch bug (hereinafter “southern chinch”), particularly in regions of the country where the southern chinch is active year-round. The southern chinch damages St. Augustinegrass by consuming the sap at the base of the leaf nodes, and applications of pesticide are normally required to control the pest. Similarly, the white grub (the larvae of May beetle or June bug) is a troublesome pest for St. Augustinegrass. The white grub damages St. Augustinegrass by devouring the roots, and can cause extensive turf damage if allowed to spread (Dudle, R. L., Id.).

Numerous varieties of St. Augustinegrass have been isolated and/or cultivated, based upon the traits that were desired in the particular variety. For example, Floratam is a variety of St. Augustinegrass that was made available in 1972. Floratam is best known for its resistance to St. Augustine decline (SAD) virus and southern chinch. Unfortunately, Floratam is a sterile variety of St. Augustinegrass, and is also intolerant to cold and shade.

The Seville variety of St. Augustinegrass is a finer-textured variety than Floratam, and possesses SAD virus and southern chinch resistance as well. Unfortunately, the Seville variety is intolerant to cold conditions.

Another variety of St. Augustinegrass is the Raleigh variety. Raleigh St. Augustinegrass is a cold tolerant, SAD virus resistant variety, which also has a higher tolerance for shade than Floratam. Unfortunately, Raleigh St. Augustinegrass is not resistant to southern chinch.

Therefore, transgenic St. Augustinegrass harboring insecticidal genes, or alternatively, genes which enhance drought and/or cold survival, are desirable in the industry, given the popularity of the species in the gulf coast region and Florida. Furthermore, a transgenic variety exhibiting the most desirable traits of the numerous St. Augustinegrass varieties would be ideal for turf application and use. In order to achieve transgenic St. Augustinegrass, a reliable and effective method of producing competent St. Augustinegrass plant cells is necessary, as well as the corresponding methods of transforming the same competent cells and regenerating plants there from.

The present invention addresses the need in the industry for a method of generating embryogenic callus capable of transformation and regeneration to viable plants. Particularly, the invention provides a method of initiating, proliferating and regenerating embryogenic callus derived from immature inflorescence explants. The invention also provides methods for the genetic transformation of embryogenic callus derived from immature inflorescence explants, and techniques for the regeneration and growth of transgenic St. Augustinegrass derived therefrom.

### BRIEF SUMMARY OF THE INVENTION

The invention is directed to a method of initiating, proliferating, and regenerating embryogenic callus from immature inflorescence explants of St. Augustinegrass (Stenotaphrum secundatum). The invention is further directed to a method of transforming and regenerating the same embryogenic callus to produce transgenic St. Augustinegrass. The use of the methods disclosed in the invention enables the generation of transgenic St. Augustinegrass expressing one or more transgenes, which may include a gene conferring resistance to a selective agent.

In a preferred embodiment, the invention provides a novel method of initiating, proliferating, transforming and regenerating embryogenic callus from immature inflorescence explants of the Floratam, Raleigh, 80-10 and 6-89-175 varieties of St. Augustinegrass, and transgenic St. Augustinegrass plants generated thereby having one or more transgenes stably integrated into the nuclear genetic material.

The transgenic St. Augustinegrass is generated using transformation methods known in the art, preferably biolistic transformation methods or Agrobacterium transformation methods, to introduce the desired transgene into the embryogenic callus. The transgene is associated with a DNA vector for introduction of the transgene.

The vector comprises a selectable marker. Selectable markers are known in the art, and may generally be any selectable marker suitable for use in St. Augustinegrass (See, for example, Miki, B., et al., [Selectable marker genes in transgenic plants: applications, alternatives and biosafety. J. Biotech. 107:193-232 (2004)]. In particularly preferred embodiments of the invention, the selectable marker is the enolpyruvylshikimate-3-phosphate synthase gene (hereinafter “CP4 gene”) or the bar gene for phosphinothricin resistance. In another particularly preferred embodiment of the invention, the selectable marker is a gene conferring resistance to the antibiotic kanamycin.

The invention further provides culture conditions and techniques for facilitating the initiation, proliferation, transformation and regeneration of embryogenic callus derived from immature inflorescence explants, as well as for the regeneration and recovery of transgenic St. Augustinegrass. The invention also provides for the use of glyphosate-containing or phosphinothricin-containing selection medium for the identification of successful transformants according to the methods of the invention.

The invention further encompasses St. Augustinegrass produced by the methods of the invention.

The features and details of the invention will be more fully appreciated in light of the following detailed description of the invention.

### BRIEF DESCRIPTION OF THE DRAWINGS

FIG. 1: Comparison of regeneration of embryogenic and non-embryogenic sectors of St. Augustinegrass cv. Floratam inflorescence explant-derived callus.

A-C: Non-embryogenic sector of Floratam inflorescence explant-derived callus, at 0, 18 and 33 days after transfer to St. Augustinegrass Regeneration (SAR) medium

D-F: Embryogenic sector of Floratam inflorescence explant-derived callus, at 0, 18, and 33 days after transfer to SAR medium.

FIG. 2: Identification of CP4-containing, Roundup®-resistant transgenic plants.

A: Calli bombarded with the CP4 gene on rooting/elongating medium containing glyphosate. Arrow indicates transgenic (glyphosate-resistant) plantlet, which is identified by its larger size, darker green color, and healthy root system in medium.

B: Transgenic, stolon-propagated plant (left) and control non-transgenic plant (right) 40 days after foliar spray with Roundup® at 128 oz/acre.

FIG. 3: Phosphinothricin (Finale™)-resistant St. Augustinegrass cv. Floratam plant derived from bar transformed inflorescence explant-derived embryogenic callus: Transgenic plant [L] and non-transgenic plant [R], 19 days after foliar application of Finale™ at 4 oz/gal.

FIG. 4: B-glucuronidase (GUS) expression in St. Augustinegrass cv. Floratam inflorescence explant-derived embryogenic callus transformed with a GUS-intron gene (pBISN1) via Agrobacterium tumefaciens. Calli were assayed for GUS expression 4 days after inoculation. (A) Callus transformed with A. tumefaciens strain KYRT1/pBISN1, cultured in Minimal medium. (B) Callus transformed with A. tumefaciens strain KYRT1/pBISN1 cultured in LB medium, (C) callus transformed with A. tumefaciens strain EHA105/pBISN1 cultured in LB medium.

### DETAILED DESCRIPTION OF PREFERRED EMBODIMENTS

I. Definitions

The following definitions are provided to facilitate understanding of certain terms used throughout the specification.

As used herein, “transgene” refers to an (initially) isolated exogenous polynucleotide that encodes at least part of a transcript or protein of interest. The exogenous polynucleotide may comprise or be operably associated with at least one heterologous regulatory element.

As used herein, “GUS” refers to β-glucuronidase, which encodes an enzyme, which has activity on various chromogenic substrates.

As used herein, a transgenic plant is a plant having one or more plant cells containing a transgene of interest, optionally in the nuclear, mitochondrial, or chloroplast genetic material, and capable of transmitting the transgene to progeny.

II. Methods of Producing Embryogenic Callus of St. Augustinegrass

Transgenic St. Augustinegrass is generated according to the following methods of the invention. More particularly, transgenic St. Augustinegrass is generated via transformation of embryogenic callus to introduce transgene(s) into the embryogenic callus, thereby enabling the regeneration of transgenic St. Augustinegrass. Importantly, embryogenic callus is generated from immature inflorescence explants, typically ranging in size from about 0.5 cm to about 3.0 cm in length. Stalks may contain up to 3 inflorescence meristems: a primary (the first to appear) and two secondary on lateral branch points. Typically, the secondary inflorescence meristems are at the smallest size range when harvested, and the primary inflorescence meristem is the largest.

Inflorescence explants harvested in this fashion are sterilized, and the outer sheaths removed using a scalpel or other fine-cutting instrument. Immature inflorescence explants are isolated from the stalk segments, and transferred intact or in segments of about 0.3 cm in length to an appropriate culture medium such as, for example, F1DG medium (See Table 1). The inflorescence explants are incubated in the dark at 25° C., and transferred to fresh medium every 4 weeks. In approximately 4 weeks, immature inflorescence explants yield calli. Calli may vary in appearance, from mucilaginous to nodular, or may be loose and/or crystalline in appearance.

Within 6 weeks, approximately 5% of starting explants have callus sectors which are observed producing masses of budlike structures, some of which may contain trichomes. These explants are sub-cultured intact to new F1DG medium. At this subculture step, the callus sectors are isolated from the original explant material. The callus is verified as embryogenic callus by its ability to proliferate, and to regenerate into plants.

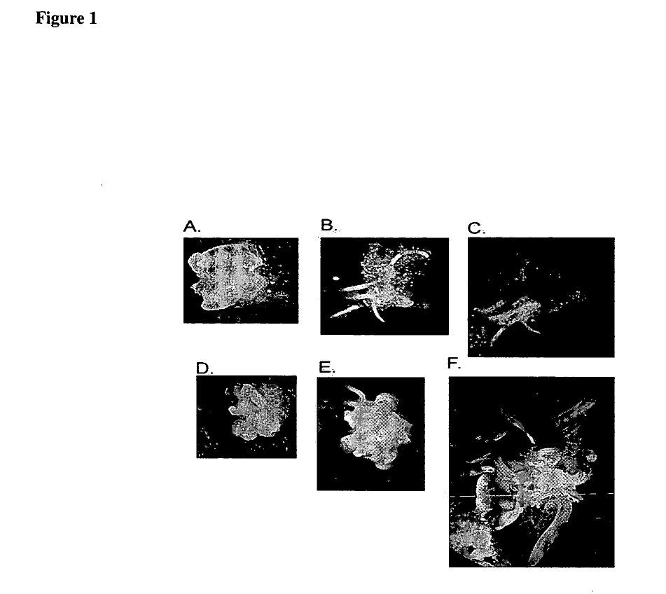

Stock callus lines are subcultured monthly, and with each subculture produce a variety of different callus types, many of which are non-embryogenic. Although embryogenic callus is heterogeneous in appearance, a typical embryogenic sector is distinguished by masses of smooth, spoon-shaped embryo structures, which may also include bud-shaped structures that appear to have juvenile leaf characteristics such as trichomes. These structures will turn green in color when placed under illumination. Embryogenic calli are verified by their ability to proliferate, and to regenerate into plants. Non-embryogenic callus tissue may appear nodular, loose, crystalline or mucilaginous in nature. The non-embryogenic nodular callus has a coarse, dull surface compared to embryogenic nodular. This is an important yet subtle distinction that is apparent to one of skill through experience and expertise in the morphology of monocot embryogenic callus. Callus in culture is typically a heterogeneous mixture of cell types, and contains both non-embryogenic and embryogenic nodular tissue that is similar in appearance (FIGS. 1A and 1D). However, when cultured on cytokinin-containing (SAR) medium (Table 5), nodular calli that is embryogenic produces green shoots (FIG. 1E, F), whereas non-embryogenic nodular calli necroses (FIG. 1B, C).

In work conducted following the teachings of the present invention, calli have been maintained in an embryogenic state for at least 3 years. The methods of the invention preferably utilize callusing medium containing 1 mg/L 2,4-D, and regeneration medium containing 2 mg/L benzyladenine.

Calli may also be maintained in an embryogenic state on medium where 0.1-0.3 mg/L benzyladenine is used in combinations with 2,4-D at 1-2 mg/L.

III. Methods of Producing Transgenic St. Augustinegrass

Embryogenic calli are transformed with a transgene in order to generate transgenic St. Augustinegrass. The transformation of embryogenic calli is achieved using transformation methods known to one of skill in the art. For example, transformation methods include, but are not limited to, microprojectile bombardment; Agrobacterium-mediated DNA transfer; the use of DNA coated silicon carbide fibers or another fiber type to move DNA into cells (‘Whiskers’ technology) (See, Kaeppler et al, Theor. Appl. Genet., 84:560-566 (1992)); PEG-mediated transformation into protoplasts (See, Negrutiu I, et al., Plant Mol Biol 8 (5):363-373 (1987)); and direct DNA uptake following electroporation of recipient protoplasts. In a preferred embodiment of the invention, microprojectile bombardment is the method used to introduce the transgene into the embryogenic callus. In a further preferred embodiment, microprojectile bombardment utilizes tungsten or gold particles, though other particles may be utilized for the purposes of microprojectile bombardment. Any appropriate biolistic (microprojectile bombardment) device may be utilized in the methods of the invention in order to achieve transformed St. Augustinegrass. Biolistic devices that may be used in the methods of the invention include, but are not limited to, the Biolistic™ PDS-1000/He (Bio-Rad Laboratories, Hercules, Calif.).

Numerous vectors may be used with the methods of the invention, provided that the vectors are suitable for plant transformation. Most commonly used vectors include pUC19, pBIN19 and derivatives of the same. Additionally, vectors used with the methods of the invention contain a selectable marker enabling the identification and isolation of successful transformants. In addition to a selectable marker gene, these vectors can contain a gene conferring a secondary trait.

Vector selectable markers useful in the methods of the invention include, but are not limited to, gene(s) conferring resistance to antibiotics such as kanamycin or hygromycin; or herbicides such as glyphosate or phosphinothricin; or gene(s) conferring the ability to use uncommon carbon sources such as mannose. In addition, selectable markers useful in the methods of the invention include gene(s) encoding for an enzyme, the presence of which may be assayed using chromogenic substrates or other identifiable means. In the particularly preferred embodiments of the invention, the selectable marker is selected from the group consisting of CP4, the bar gene and the nptII gene.

In another preferred embodiment of the invention, Agrobacterium tumefaciens transformation is the method used to introduce the transgene into the embryogenic callus. Agrobacterium tumefaciens strains that can be used with the methods of the invention include, but are not limited to, Agrobacterium tumefaciens strains EHA105, KYRT1, LBA4404 and ABI. Some recombinant vectors include, but are not limited to pBISN1 (See Narasimhulu S. B., et al., The Plant Cell, 8:873-886 (1996)), pBIN19 and pBI121. The transgenes used may include, but are not limited to, those described in Section IV infra. The selectable markers used may include, but are not limited to, the reporter gene GUS and genes conferring resistance to kanamycin, hygromycin, glyphosate, phosphinothricin.

The methods of the invention are applicable to all varieties of St. Augustinegrass, including but not limited to Floratine, Bitter Blue, Floratam, Seville, Raleigh, Texas Common, Palmetto, Delmar, dwarf line 80-10, dwarf line 6-89-175, Garrets 141, Jade, Woerner's classic, Salzman, Mercedes, and dwarf line 6-89-196. In a preferred embodiment of the invention, the methods are applied to Delmar, Raleigh, Floratam, Seville, dwarf line 80-10, dwarf line 6-89-175, and dwarf line 6-89-196 varieties. In a particularly preferred embodiment of the invention, the methods are applied to Floratam, 80-10, 6-89-175 and Raleigh varieties.

Successful transformants are regenerated into complete St. Augustinegrass, and optionally may have the transgene stably integrated into the nuclear, mitochondrial or chloroplast genetic material. Successful transformants are capable of transferring the transgene to progeny. St. Augustinegrass progeny are generated using techniques known in the art, and include but are not limited to, vegetative or asexual reproduction. For example, St. Augustinegrass is commercially propagated clonally (as sod), and is rarely sold or propagated through seed-derived progeny. Nonetheless, the invention further contemplates the generation of St. Augustinegrass progeny through sexual reproduction. Furthermore, non-sterile varieties of St. Augustinegrass regenerated from successful transformants are capable of transferring the transgene to the seeds and/or progeny derived through sexual reproduction.

The methods of the invention are particularly appropriate for the generation of transgenic St. Augustinegrass of the Floratam variety. The Floratam variety of St. Augustinegrass is a highly important commercial variety of St. Augustinegrass used in the United States, but the variety produces male-sterile flowers. Therefore, embryogenic callus cannot be derived from seeds of this variety, as no seeds are produced. Accordingly, the methods of the invention provide a means for generating transgenic St. Augustinegrass of asexually-reproduced varieties that produce male-sterile flowers, including but not limited to the Floratam variety.

IV. Transgenes Useful for the Transformation Methods of the Invention

The methods of the invention enable transformation of St. Augustinegrass plants with transgene(s). In a preferred embodiment of the invention, the gene is an exogenous transgene. Using the methods of the invention, St. Augustinegrass embryogenic callus may be transformed with the following transgenes.

In order to improve the resistance to environmental stresses, the following transgenes may be introduced into St. Augustinegrass embryogenic callus using the methods of the invention. Environmental stresses include, but are not limited to, temperature extremes, drought and salinity. Transgenes include, but are not limited to those genes recited in the following paragraphs.

Genes which confer cold tolerance such as the E. coli MnSOD gene; the CAP85 and CAP160 genes of spinach; the soybean SCOF-1 gene; the Arabidopsis CBF3 gene; the barley BLT4 gene; the Arabidopsis GPAT gene; the Athrobacter globiformis gene for choline oxidase (cod A); CBF1 and CBF4 genes from Arabidopsis; CAT3 gene from maize and CAT1 gene from tomato; and DREB1A gene. In a preferred embodiment of the invention, genes that provide cold tolerance comprise, or alternatively consist of, the CBF1, CBF4, DREB1A, and E. coli MnSOD genes.

Genes that confer drought resistance such as the turgor responsive gene trg 31; the bacterial fructan genes; the δ-Pyrroline-5-Carboxylate Synthetase gene; the barley HVA1 gene; the Arabidopsis ERD1 gene; the mannitol-1-P dehydrogenase gene; NtC7 gene of tobacco; glutamine synthetase (GS) gene of rice; OsCDPL7 gene of rice; the DRO2 gene, the E. coli trehalose synthesis genes (TPSP/TPP); and the DREB2A gene. In a preferred embodiment of the invention, genes that confer drought resistance comprise, or alternatively consist of, the DRO2, DREB2A, and trehalose synthesis genes.

Transgenes which enable the modification of plant phenotypic characteristics, such as for example, plant color and size (e.g., genes controlling dwarfism), include but are not limited to, the 2-oxidase gene; the OsGA20-ox2 (See, Sasaki A., et al., Nature, 416:701-702 (2002)); and OsGA3-ox2 genes from rice (See, Itoh H., et al., PNAS USA, 98:8909-8914 (2001)); the BAS1 gene (See, Neff, M M., et al., PNAS USA, 96(26):15316-15323 (1999)); the rol (A, B, and C) genes; the phyA gene; the crtO gene; the lycopene cyclase gene; OsMADS45 gene; and OsMADS1 gene. In a preferred embodiment of the invention, genes that confer dwarfing phenotype comprise, or alternatively consist of, the BAS1 and 2-oxidase genes.

Transgenes that confer herbicide resistance, such as for example, the CP4 gene, the bar gene, and the pat gene. In a preferred embodiment of the invention, genes that confer herbicide resistance comprise, or alternatively consist of, the CP4, bar, and pat genes.

St. Augustinegrass is susceptible to a number of pathogen, fungal and viral-mediated infestations. For example, St. Augustinegrass is susceptible to southern lawn chinch bug, White grubs, sod webworms, armyworms, cutworms, fungal infections such as brown patch, gray leaf spot, Helminthosporium, Pythium, rust, and downy mildew, as well as viral infections such as SAD virus. Accordingly, general transgenes include genes useful in combating the above-listed diseases of St. Augustinegrass.

More specifically, transgenes that are useful in combating infection and inhibiting St. Augustinegrass pests include, but are not limited to the genes recited in the following paragraphs:

Genes which deter insect feeding such as the Phaseolus vulgaris alpha amylase inhibitor and Arcelin 5A seed storage genes; the sweet potato trypsin inhibitor; the Bacillis thuringiensis cry1A and cry1B genes; the Nicotiana alata proteinase inhibitor gene; the Mir1 cysteine proteinase inhibitor; and the chitinase gene. In a preferred embodiment of the invention, genes that deter insect feeding comprise, or alternatively consist of, the Bacillis thuringiensis cry1A and cry1B genes.

Approaches that confer virus resistance in plants include silencing of viral nucleotides. Also, the use of genes such as viral coat protein genes; viral NSM genes; viral antisense RNA genes; and viral nuclear inclusion genes has been demonstrated to increase resistance to viral pathogens.

Genes that confer microbial pathogen resistance such as the beta 1,3-glucanase gene; the cecropin gene; the MeRIP gene of Mirabilis expansisn; the chitinase gene and various antimicrobial peptide genes.

Additional genes useful in conferring disease resistance include, but are not limited to, Rpg1 gene of barley; NDR1 gene of Arabidopsis and various R genes and combinations of R genes from the superfamilies NB-LRR, eLRR and LRR-kinase. The expression of genes for transcription factors (TF) that act on genes that are directly involved in disease resistance. Such TF include Pti 4,5,6 and those described in U.S. Pat. No. 6,664,446, which is incorporated herein in its entirety. In a preferred embodiment of the invention, genes that confer disease resistance comprise, or alternatively consist of, the R genes and transcription factors that act on genes that are directly involved in disease resistance.

Additional general transgenes useful in the methods of the invention include, but are not limited to, genes that improve nutrient utilization; genes that improve nutritional content; genes that improve shade tolerance; genes which include regulatory elements enabling the control of the same gene as well as endogenous gene(s); genes that influence the production of allergenic pollen; genes that induce male sterility (such as the CKX1 gene and the GAI gene; the Arabidopsis EMF1 and 2 genes); genes that render the plants useful for phyto-remediation or production of Plant-Made-Pharmaceuticals or their components; genes that improve forage quality; and combinations of any of the aforesaid transgenes.

Transgenes include the use of promoters for transforming St. Augustinegrass, which could be used in concert with other transgenes. These promoters include, but are not limited to, the promoters recited in the following paragraphs:

Constitutive plant promoters include the cauliflower mosaic virus (CaMV) 35 S promoter, the maize ubiquitin promoter, the figwort mosaic virus promoter (FMV), the nopaline synthase promoter, and the rice actin promoter (OsAct1) (See Christensen, A. H., et al., Ubiquitin promoter-based vectors for high-level expression of selectable and/or screenable marker genes in monocotyledonous plants. Transgenic Res, 5: 213-218 (1996));
Drought-responsive promoters such as the promoters controlling the rd29A gene of Arabidopsis, the Ppc1 gene of the ice plant, the blt4 gene of barley, and the CPRD genes of cowpea;

Wound-inducible promoters (for insect defense genes) such as the promoters controlling the POT9 gene of poplar, or TCH2 gene of Arabidopsis;

Senescence promoters such as the promoters driving senescence-associated genes (SAGs) (See, Dang, J. L. et al., Senescence and Programmed Cell Death, Chap. 20 (pp. 1044-1100) in ‘Biochemistry & Molecular Biology of Plants’, edited by Buchanan, B B, Gruissem, W., and Jones, R L. Published by The American Society of Plant Physiologists, Rockville, Md. (2000)) which is incorporated herein by reference;

Osmotic stress response promoters such as the promoter controlling the WAK14 gene of Arabidopsis; and

Tissue specific promoters where expression of the gene linked to the promoter occurs only or preferentially in selected tissues. [See, for example, Koziel, M. G., et al. (1993). Field performance of elite transgenic maize plants expressing an insecticidal protein derived from Bacillus thuringiensis. BioTechnology 11:194-200 (1993); Wong, E. Y., et al., Arabidopsis thialiana small subunit leader and transit peptide enhance expression of Bacillus thuringiensis proteins in transgenic plants. Plant Mol. Biol., 20:81-93 (1992); Kyozuka, J., et al., Light-regulated and cell-specific expression of Tomato rbcS-gusA and Rice rbcS-gusA fusion genes in transgenic rice, Plant Physiology 102(3):991-1000 (1993); Hudspeth, R. L., et al., Structure and expression of maize gene encoding the phosphoenolpyruvate carboxylase isozyme involved in C4 photosynthesis, Plant Mol. Biol., 579-589 (1989); and Estruch, J. J., et al., The expression of a synthetic Cry1Ac gene in maize confers resistance to European corn borer. In: Proceedings, Insect resistant maize: Recent advances and utilisation, International Wheat and Maize Research Institute (CIMMYT), Mexico City, Mexico. pp. 172-174 (11/27-12/3, 1994)]. Such promoters include PHT1 a root specific promoter and the R1-MAS promoter yielding strong expression in roots. Promoters from genes specific to pollen such as ZMG13 and AtSTP6 (See Scholz-Starke, J., et al. Plant Physiol, 131:70-77 (2003)) or specific to the tapetum such as the TAP promoter (Mariana C., et al., Nature, 347:737 (1990)). Pollen active promoters include PTA29, PTA26 and PTA13. ACP a promoter specific to both pollen and ovule. Promoters that confer mesophyll-specific expression such as the ribulose-bisphosphate carboxylase small subunit promoters. Additional tissue-specific promoters include, but are not limited to, the A thal EFIα promoter that protects reproductive parts from Round-up®; Root-specific promoter PHT1; Maize seed-specific glutamine synthetase (GS[1-2]) promoter; OsGA3ox2 promoter specific for vegetative (nonreproductive) tissue (See, Sakamoto T., et al., Nature Biotech., 21 (8):909-913 (2003)); the AtWRKY18 promoter; the R1-MAS promoter; and the MEA promoter.

### V. EXAMPLES

The following examples are provided to more particularly describe the invention, and are not to be construed as limiting the invention to particular applications, embodiments, or varieties of St. Augustinegrass plants.

### Example 1

### Maintenance of Stock Plants and Harvesting of Inflorescence Explants

Stock plants were maintained in soil (Metro Mix™ 360) in 6″ and 8″ pots under ambient greenhouse conditions. Plants were fertilized weekly with Peters™ CalMag 15-5-15, monthly with Scotts Turfbuilder™ and every three (3) months with Ironite™.

Inflorescence explants were harvested from established stock plants between the months of December and May. St. Augustinegrass has a rigid flowering season that cannot be artificially altered (that fact has an important association with the longevity of the callus once established). St. Augustinegrass stalks may contain up to 3 inflorescence meristems: a primary (the first to appear) and two secondary on lateral branch points. Typically, the secondary inflorescence is at the smallest size range when harvested, and the primary is the largest. All inflorescence explants were used for culturing, and any could initiate embryogenic callus.

### Example 2

### Preparation of Explants

Inflorescence explants harvested in Example 1 were stored in resealable plastic bags at 4° C. until they were prepared for culturing. Inflorescence explants stored in this fashion can be stored for at least 10 days and still yield viable embryogenic callus.

Inflorescence explants used in the experiment were sterilized as follows: excess leaves and stems were trimmed back or removed, leaving at least one outer leaf sheath. Inflorescence explants were then washed with a 5% solution of soap (Sparkleen™) for 5 minutes. Following washing, inflorescence explants were incubated for 2 minutes in 70% ethanol, and 10 minutes in 15% Chlorox™ containing 0.01% detergent (Triton-X or Tween-20), during which they were treated with vacuum pressure 5 minutes to allow the bleach or detergent to infiltrate. Inflorescence explants were then rinsed three (3) times in sterile water, dry blotted, and dissected for culture under a microscope.

The outer sheath was removed from the inflorescence explants using a No. 11 scalpel and fine-tipped forceps. One of skill in the art has the experience to remove the outer sheath of inflorescence explant tissue as prepared above. The inflorescence explant was then isolated from the sheath, and transferred [intact (if <1 cm long) or as 0.3 cm-long sections (if >1 cm long)] to F1DG medium (Table 1). Cultures were placed in the dark, incubated at 25° C., and subcultured every 4 weeks.

TABLE 1
F1DG Medium Composition¹

| Component | Weight (per liter) | |
|---|---|---|
| MS Salts | 1X | |
| MS Vitamins² | 1X | |
| Sucrose | 30 | g |
| Casein Hydrolysate | 0.5 | g |
| Proline | 1.5 | g |
| MES buffer | 0.5 | g |
| 2,4-dichlorophenoxyacetic acid (2,4-D)² | 1.0 | mg |
| Gelrite | 3.0 | g |

¹pH adjusted to 5.8
²MS Vitamins and 2,4-D were added after autoclaving

TABLE 2
MS1DG Medium Composition¹

| Component | Weight (per liter) | |
|---|---|---|
| MS Salts | 1X | |
| MS Vitamins | 1X | |
| Sucrose | 30 | g |
| 2,4-dichlorophenoxyacetic acid (2,4-D) | 1.0 | mg |
| Gelrite | 3.0 | g |

¹pH adjusted to 5.8

After approximately 4-5 weeks of incubation, immature inflorescence explants yielding calli were examined. Callus types typically vary from mucilaginous to nodular, to loose/chrystalline. However, by 6 weeks, on approximately 5% of starting explants, callus sectors were observed producing masses of budlike structures, some of which may contain trichomes. These explants were sub-cultured intact to new F1DG medium (Table 1). At this subculture step, the callus sectors were isolated from the original explant material. The callus was verified as embryogenic callus by its ability to proliferate, and to regenerate into plants.

### Example 3

### Alternative Initiation Media

Culture media with different composition than F1DG was used to successfully initiate embryogenic calli from St. Augustinegrass inflorescence explants. These media include: MS1DA (same as MS1DG (Table 2) except solidified with 1% agar instead of 0.3% Gelrite), MS1DPCH (Table 3), MS5DPCH (Table 4), MS1DPCH solidified with 1% agar instead of 0.3% Gelrite, and MS5DPCH solidified with 1% agar instead of 0.3% Gelrite.

TABLE 3
MS1DPCH medium composition¹

| Component | Weight (per liter) | |
|---|---|---|
| MS salts | 1X | |
| MS vitamins | 1X | |
| Sucrose | 30 | g |
| Proline | 1.5 | g |
| Casein hydrolysate | 0.5 | g |
| MES buffer | 0.5 | g |
| 2,4-dichlorophenoxyacetic acid | 1.0 | mg |
| Gelrite | 3.0 | g |

¹pH adjusted to 5.8

TABLE 4
MS5DPCH medium composition¹

| Component | Weight (per liter) | |
|---|---|---|
| MS salts | 1X | |
| MS vitamins | 1X | |
| Sucrose | 30 | g |
| Proline | 1.5 | g |
| Casein hydrolysate | 0.5 | g |
| MES buffer | 0.5 | g |
| 2,4-dichlorophenoxyacetic acid | 5.0 | mg |
| Gelrite | 3.0 | g |

¹pH adjusted to 5.8

### Example 4

### Maintenance of Embryogenic Callus

Subculturing monthly to F1DG medium propagates embryogenic calli. Stock calli were stored in the dark at 24-28° C. temperature. Typical embryogenic callus is heterogeneous: a typical embryogenic sector is distinguished by masses of smooth, spoon-shaped embryo structures, which may also include bud-shaped structures that appear to have juvenile leaf characteristics such as trichomes, which will green up when calli are placed under illumination. Furthermore, non-embryogenic calli can also be produced on the same callus segment, and non-embryogenic calli appear mucilaginous, nodular, or loose/chrystalline in nature. Non-embryogenic calli sectors were removed and discarded during the sub-culturing. The longevity of embryogenic calli is not yet known; however, a 3 year-old callus line that is maintained under the same conditions described above still continues to propagate and regenerate normally.

Other media useful for maintenance of embryogenic callus include, for example, F1DG with various combinations of 2,4-D and benzyl-adenine (hereinafter “BA”) instead of 1 mg/L 2,4-D alone. Alternatively, the hormone combinations could include 1-2 mg/L 2,4-D in combinations with 0.1-0.3 mg/L BA.

### Example 5

### Regeneration of Embryogenic Callus

In order to regenerate embryogenic calli into plants, sectors of embryogenic calli were transferred to St. Augustinegrass Regeneration (SAR) medium (Table 5).

TABLE 5
SAR Medium Composition¹

| Component | Weight (per liter) | |
|---|---|---|
| MS Salts | 1X | |
| MS Vitamins | 1X | |
| Sucrose | 30 | g |
| Benzyladenine | 2.0 | mg |
| Gelrite | 3.0 | g |

¹pH adjusted to 5.8

Calli were incubated in the dark at 24-28° C. temperature for approximately three (3) weeks. Following incubation, embryogenic calli were typically converted to trichome-covered shoot-like structures. Plates were then transferred to illumination [sixteen (16) hours of light, followed by eight (8) hours of darkness] at 25° C. Under these conditions, the shoot-like structures rapidly turn green. Plates were maintained under these illumination conditions for two (2) weeks. At the end of the duration of illumination on SAR medium (Table 5), calli were transferred to MSO medium (Table 6) in deeper plates (1.5 cm plates), and maintained under illumination. Within three (3) to four (4) weeks, shoot-like structures become rooted plantlets, which can be subdivided and transferred directly to soil in the greenhouse.

TABLE 6
MSO (Hormone-free Rooting/Elongation) Medium Composition¹

| Component | Weight (per liter) | |
|---|---|---|
| MS Salts | 1X | |
| MS Vitamins² | 1X | |
| Sucrose | 30 | g |
| Gelrite | 3.0 | g |

¹pH adjusted to 5.8

### Example 6

### Generation of CP4-Transformed St. Augustinegrass Embryogenic Calli

Calli were maintained in the dark for 4-24 months, at 24-28° C. on F1DG medium (Table 1), with monthly transfers. For purposes of the generation of transformed St. Augustinegrass embryogenic calli, biolistic (particle bombardment) techniques for transformation were utilized. Calli were transferred as approximately 0.1-0.3 cm segments to filter papers on F1DG (Table 1) or MS 1DG medium (Table 2). Prior to bombardment, calli were plasmolyzed for 4-6 hours to overnight on medium supplemented with osmoticum, typically 0.25 M mannitol or 0.25 M mannitol+0.25 M sorbitol. Calli were placed on a microprojectile shelf, which gave the microprojectile a traveling distance of 5.5 cm. Calli were bombarded two (2) times or three (3) times, at either 900 or 1,200 psi using a Biolistic™ PDS-1000/He, with microprojectiles coated with appropriate vector containing the gene(s) of interest. Calli were transferred twenty-four (24) hours later to osmoticum-free medium, and within six (6) days the calli were subdivided into approximately two (2) mm segments. The segments were transferred to F1DG (Table 1) or MS1DG medium (Table 2) containing a selection agent.

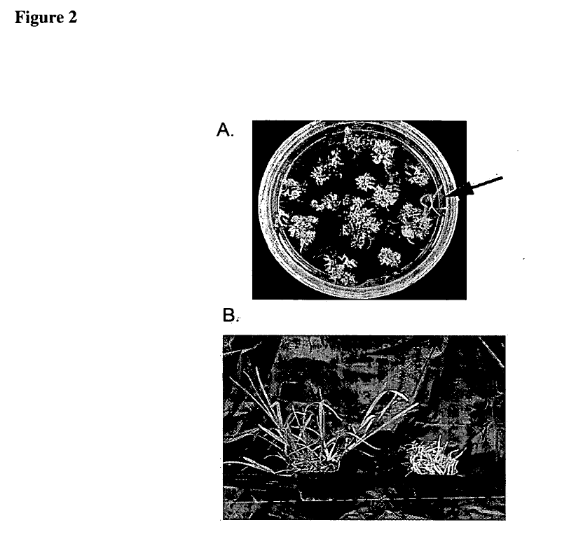

The calli were maintained for four (4) to eight (8) weeks in the dark, after which time all surviving embryogenic sectors were transferred to regeneration (SAR) medium (Table 5) containing a selection agent. The calli were maintained on this medium for five (5) weeks. At the end of the third (3) week, calli produced etiolated shoots and shoot buds, and were moved into illumination having a sixteen (16) hour light, eight (8) hour dark photoperiod. At the end of the fifth (5) week on SAR medium containing a selection agent, calli were moved to hormone-free rooting/elongation (MSO) (Table 6) medium containing a selection agent. If the selection agent was glyphosate, the concentration was reduced by 80% from the MS1DG medium (Table 2) to the SAR (Table 5) medium, and again by 60% from the SAR (Table 5) medium to MSO medium (Table 6). Transgenic shoots were identified as darker-green, with healthy roots present in the medium (FIG. 2A).

### Example 7

### Verification of Transformed, Glyphosate-Tolerant Shoots

Shoots from potentially transgenic plantlets were subjected to testing for the expression of the CP4 gene. A 2-3 mm piece of leaf from the developing shoots was removed and placed in a 1.5 ml Eppendorf microfuge tube. A 400 μl aliquot of water was added to the tube containing the leaf sample. The leaf tissue was ground in the sample buffer using a clean plastic applicator (Puritan, Guilford, Me.). A CP4 strip (RUR-HS Test Kit, Strategic Diagnostics, Inc., Newark, Del.) was inserted into the tube containing the leaf sample and the sample buffer. After 5 minutes two pink bands on the strip indicated the presence of the CP4 protein. A single pink band indicated the absence of the CP4 protein.

### Example 8

### Verification of Roundup®-Resistant Phenotype in Greenhouse-Grown Transgenic Plants

Plants were node-propagated by rooting a cutting from a stolon. Once established, that node-propagated individual was sprayed with Roundup® at a rate of 128 oz/acre. Roundup® resistance of transgenic lines was scored over a 5 week period on a numerical scale: 1=dead, 5=undamaged (FIG. 2B).

In addition to Floratam, varieties 80-10, 6-89-175 and Raleigh were transformed with the CP4 gene. Table 7 describes the number of filters bombarded, transgenic shoots recovered based on screening with CP4 strips and the number of transgenic shoots that were resistant to Roundup®.

TABLE 7
Summary of the Roundup ® resistant St. Augustinegrass
plants derived from biolistic transformation experiments

| | # samples | # CP4 + transgenic | # Roundup ®- |
|---|---|---|---|
| Cultivar | bombarded | shoots recovered | resistant lines |
| Floratam | 94 | 37 | 18 |
| 80-10 | 77 | 7 | 3 |
| 6-89-175 | 23 | 2 | 1 |
| Raleigh | 60 | 12 | not tested* |

*Not yet tested as of time of filing of this application.

### Example 9

### GUS Activity

Utilizing the St. Augustinegrass embryogenic calli transformation techniques of Example 6 supra, calli were transformed with vectors incorporating a GUS gene as an additional selectable marker. Following transformation, the following example was used to test for the presence of the transgene. Leaves from transgenic St. Augustinegrass were cut into cross-sections and stained for GUS activity in histochemical staining solution with 100 mM ascorbic acid, 100 mM sodium phosphate buffer pH 7.0, 1 mg/mL of X-Gluc (5-bromo-4-chloro-3-indoyl glucuronide, Sigma #B650) and 0.1% (v/v) TRITON X-100® (Union Carbide). After incubation overnight at 37° C., chromogenic intensity was rated on a qualitative scale from “very strong” to “very weak” to “none”. Non-transgenic St. Augustinegrass plants did not display detectable GUS activity.

### Example 10—Phosphinothricin activity

Utilizing the St. Augustinegrass embryogenic calli transformation techniques of Example 6 supra, calli were transformed with vectors incorporating a gene for phosphinothricin resistance. Calli were transferred 6 days after bombardment to MS1DG (Table 2) medium containing 5 mg/L of phosphinothricin. After 6 weeks, calli were transferred to SAR (Table 5) medium, containing 5 mg/L of phosphinothricin, on which they were maintained for 4-6 weeks. Green shooting calli were then transferred to MSO (Table 6)+5 mg/L of phosphinothricin, and placed under a 16H:8H (light:dark) photoperiod.

### Example 11

### Liberty-Link Screening

Potential transgenic plantlets from Example 10 were identified as rooting on MSO (Table 6)+5 mg/L of phosphinothricin. Plants were isolated, and transferred to individual test tubes containing MSO (Table 6)+5 mg/L of phosphinothricin, and chlorophenol red (pH indicator) dye. If a plant was not transgenic, the dye turned deep pink with the secretion of ammonia from the roots in response to the phosphinothricin. Transgenic plants turned the media yellow. Plants were transferred to soil in the greenhouse, and were cloned twice by nodal propagation. A nodal clone of a potential transgenic plant was sprayed with Finale™ at 4.0 oz/gal, along with a nodal clone of a control (non-transgenic) plant from the same cell line. Plant appearance was then monitored up to 4 weeks after spraying, and survival was scored numerically, with 1=total death and 5=undamaged.

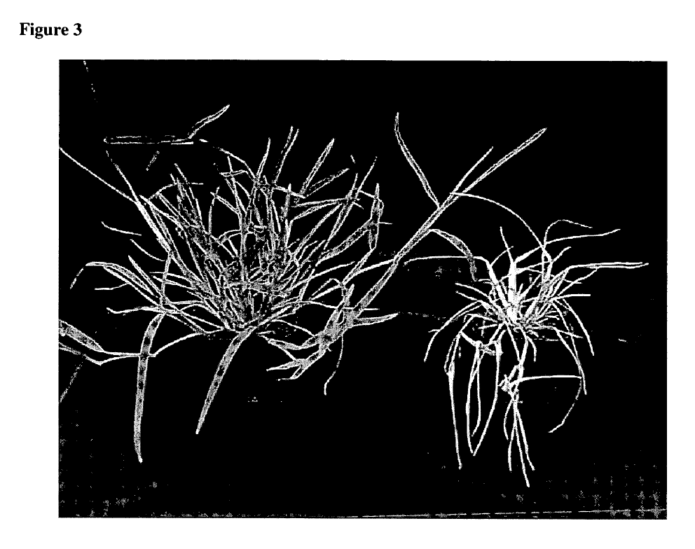

Fifty-seven transgenic Floratam plants were acclimated to the greenhouse and nodal clones of these plants were sprayed with Finale™ at 4.0 oz/gal. Twenty-five of these lines were highly resistant to phosphinothricin and demonstrated slight to no visible spray damage (FIG. 3).

### Example 12

### Transformation of St. Augustinegrass Inflorescence Explant-Derived Embryogenic Calli Using Agrobacterium tumefaciens

Agrobacterium tumefaciens strains EHA105 and KYRT1 containing binary vectors for kanamycin resistance were cultured overnight at 28° C. in 1 mL of liquid LB (Luria Bertani) broth (GibcoBRL cat. No. 12780-052) or M9 minimal medium, that contained the appropriate antibiotics. (example: EHA105 use 50/50 mg/L rifampicin/kanamycin). These strains both contained the vector pBISN1 carrying the nptII gene for kanamycin resistance.

Culture samples were used the following day to inoculate 100 mL of the same medium plus antibiotics, to which was added acetosyringone to 20-40 mg/L. Cultures were incubated with shaking at 28° C. until the suspension reached an OD₆₀₀ of 0.1-0.4. Agrobacterium tumefaciens suspensions were then pelleted by centrifugation, and resuspended in liquid MS1D (Table 2, MS1DG without Gelrite) containing 20-40 mg/L acetosyringone.

Inflorescence explant-derived embryogenic Floratam callus were selected for transformation from stock cultures (embryogenic sectors only). Embryogenic sectors of 3-4 mm diameter in size were picked from the stock callus and immersed in the resuspended bacteria. Callus may be wounded or coarsely chopped prior to immersion, to stimulate Agrobacterium interaction, and surface cell proliferation. The callus/bacteria suspension was placed on a slow shaker for 30 minutes. Afterward, the bacterial suspension was drawn off.

Calli were blotted on sterile filter paper and then transferred to sterile filter paper (Whatman no. 2) on solidified co-culture medium [MS1DG (Table 2) containing 10 g/L glucose, 0.5 g/L casein hydrolysate and 20-40 mg/L acetosyringone]. Co-culture medium may also contain thiol compounds such as cysteine or dithiothreitol to enhance viability. Plates were then incubated in the dark at 21-28° C. for several days. Calli were transferred to sterile multi-well plates and washed for 15 minutes in curing medium (liquid MSO medium containing 250 mg/L cefotaxime) while shaking gently.

Afterward, calli were removed from the wells, blotted dry as before, and transferred to callus initiation medium (F1DG containing 250 mg/L cefotaxime, and 100 mg/L kanamycin). Calli were cultured on this medium for 6 weeks at 28° C., with weekly subculturing.

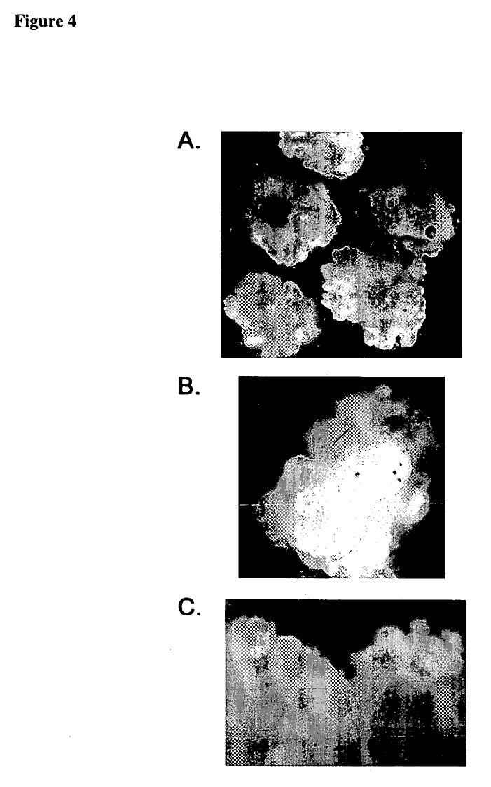

Immediately following co-culture, a subset of calli were stained for GUS activity in histochemical staining solution with 100 mM ascorbic acid, 100 mM sodium phosphate buffer pH 7.0, 1 mg/mL of X-Gluc (5-bromo-4-chloro-3-indoyl glucuronide, Sigma #B650) and 0.1% (v/v) TRITON X-100® (Union Carbide). After incubation overnight at 37° C., chromogenic intensity was scored (Table 8). Blue spots were observed on calli inoculated with strain KYRT1 and EHA105 (See FIG. 4).

This experiment compared the ability of 2 strains of Agrobacterium, EHA105 and KYRT1 (each containing pBISN1) to transform St. Augustinegrass. Agrobacterium cultures were grown in either LB or minimal medium. Calli were GUS-stained 4 days after inoculation. Preliminary GUS data indicates that the KYRT1 strain yielded higher transformation in St. Augustinegrass cv. Floratam than strain EHA105.

TABLE 8
Results of preliminary GUS test of St. Augustinegrass cv. Floratam
inflorescence explant-derived embryogenic calli, transformed via
Agrobacterium tumefaciens.

| Strain: | Agro medium: | Plasmid: | # calli tested | # GUS foci |
|---|---|---|---|---|
| EHA105 | Minimal | pBISN1 | 6 | 6 |
| EHA105 | LB | pBISN1 | 8 | 11 |
| KYRT1 | Minimal | pBISN1 | 6 | 50 |
| KYRT1 | LB | pBISN1 | 6 | 110 |

After the 6 week callus selection period, calli are transferred (viable embryogenic sectors only) to solidified regeneration/selection medium [(SAR medium (Table 5) containing antibiotics (100 mg/L Timentin or 200 mg/L cefotaxime), and 50 mg/L kanamycin)].

Cultures are incubated at 28° C. in the dark for 3 weeks, with weekly subculturing. Cultures are then transferred to illumination (16H/8H: light/dark) at 23° C. for another 3 weeks. Afterward, greening sectors are transferred to solidified rooting/selection medium [MSO medium (Table 6) containing antibiotics (100 mg/L Timentin or 200 mg/L cefotaxime), and 20 mg/L kanamycin]. Dark-green rooting shoots are then assayed for the presence of the transgene.

The invention may be practiced in ways other than those particularly described in the foregoing description and examples. Numerous modifications and variations of the invention are possible in light of the above teachings and, therefore, are within the scope of the appended claims.

The entire disclosure of each document cited (including patents, patent applications, journal articles, abstracts, manuals, books, or other disclosures) in the Background of the Invention, Detailed Description, and Examples is herein incorporated by reference in their entireties.

1. A method of producing embryogenic callus from immature inflorescence explants of St. Augustinegrass plants, comprising:
a) culturing an explant of St. Augustinegrass immature inflorescence to initiate growth of callus tissue; and
b) isolating embryogenic callus which is regenerable into a St. Augustinegrass plant.

2. The method of claim 1, wherein the isolated embryogenic callus is regenerable into an adult St. Augustinegrass plant.

3. The method of claim 1, wherein the isolated embryogenic callus is regenerable into a fertile St. Augustinegrass plant.

4. The method of claim 1, wherein the isolated embryogenic callus is genetically identical to the source from which the explant was obtained.

5. The method of claim 1, wherein the isolated embryogenic callus is regenerable into an adult St. Augustinegrass plant that is genetically identical to the source from which the explant was obtained.

6. The method of claim 1, wherein the immature inflorescence are about 0.5 to about 3.0 centimeters in length at the time of explant harvesting.

7. The method of claim 1, wherein the immature inflorescence-explant is cultured on F1DG callus initiation medium.

8. The method of claim 7, wherein the F1DG callus initiation medium includes proline.

9. The method of claim 1, wherein the steps of culturing the explant to initiate growth of callus and maintaining the embryogenic callus are both carried out in a medium comprising an identical hormone composition.

10. Isolated embryogenic callus produced by the method of claim 3.

11. A method of regenerating St. Augustinegrass plants from immature inflorescence explants, comprising:
a) culturing an explant of St. Augustinegrass immature inflorescences to initiate growth of callus tissue;
b) isolating embryogenic callus from the cultured explant; and
c) regenerating the embryogenic callus into a St. Augustinegrass plant.

12. The method of claim 11, wherein the regenerated St. Augustinegrass plant is an adult plant.

13. The method of claim 11, wherein the regenerated St. Augustinegrass plant is fertile.

14. The method of claim 11, wherein the regenerated St. Augustinegrass plant is genetically identical to the source from which the explant was obtained.

15. The method of claim 11, wherein the immature inflorescence are about 0.5 to about 3.0 centimeters in length at the time of explant harvesting.

16. The method of claim 11, wherein the immature inflorescence explant is cultured in F1DG callus initiation medium.

17. The method of claim 16, wherein the F1DG callus initiation medium includes proline.

18. The method of claim 11, wherein the embryogenic callus is regenerated on SAR regeneration medium.

19. A method of generating transformed St. Augustinegrass plants, comprising:
a) harvesting St. Augustinegrass immature inflorescences explant tissue;
b) culturing the explant tissue to initiate callus tissue growth;
c) isolating embryogenic callus from the explant tissue;
d) transforming the callus tissue with a DNA vector comprising a transgene(s);
e) isolating the transformed callus; and
f) regenerating the transformed callus into a St. Augustinegrass plant.

20. The method of claim 19, wherein the transformed callus is regenerated into mature transgenic St. Augustinegrass plants.

21. The method of claim 19, wherein the transgenic St. Augustinegrass plants can be reproduced by sexual or asexual means.

22. The method of claim 19, wherein the immature inflorescence from which explant tissue is harvested are about 0.5 to about 3.0 centimeters in length at the time of explant harvesting.

23. The method of claim 19, wherein the immature inflorescence explant tissue is cultured on F1DG callus initiation medium.

24. The method of claim 20, wherein the transformed callus is regenerated on SAR regeneration medium.

25. The method of claim 19, wherein the transformed callus is isolated on selection medium comprising a selective agent.

26. A transgenic St. Augustinegrass plant produced by the method of claim 19, the St. Augustinegrass plant comprising a transgene stably integrated into its nuclear genetic material.

27. The transgenic St. Augustinegrass plant of claim 26, wherein the variety of transgenic St. Augustinegrass is selected from the group consisting of Floratine; Bitter Blue; Floratam; Seville; Raleigh; Texas Common; Delmar; Dwarf line 80-10; Dwarf line 6-89-175; Garrets 141; Jade; Woerner's classic; Salzman; Mercedes; Dwarf line 6-89-196; or any combination thereof.

28. The transgenic St. Augustinegrass plant of claim 27, wherein the variety of transgenic St. Augustinegrass is Floratam or Raleigh.

29. The transgenic St. Augustinegrass plant of claim 26, wherein the transgenic St. Augustinegrass plant can transmit the transgene to progeny.

30. The transgenic St. Augustinegrass plant of claim 29, wherein the St. Augustinegrass plant is propagated asexually.

31. The method of claim 19, wherein the transgene conveying improved resistance to an herbicide is selected from the group consisting of: CP4 epsps, bar and pat, or any combination thereof.

32. The method of claim 19, wherein the transgene conveys a phenotype selected from the group consisting of improved resistance to temperature extremes; improved resistance to herbicides; improved resistance to drought; improved shade tolerance; altered plant coloration; altered plant size; improved resistance to fungal, bacterial and viral infection; improved resistance to pests; or any combination thereof.

33. The method of claim 32, wherein the transgene conveys the phenotype of improved resistance to herbicides.

34. The method of claim 32, wherein the transgene conveying improved resistance to temperature extremes is selected from the group consisting of the E. coli MnSOD gene; the CAP85 and CAP160 genes of spinach; the soybean SCOF-1 gene; the Arabidopsis CBF3 gene; the barley BLT4 gene; the Arabidopsis GPAT gene; the Athrobacter globiformis gene for choline oxidase (cod A); CBF1 and CBF4 genes from Arabidopsis; CAT3 gene from maize and CAT1 gene from tomato; DREB1A gene; or any combination thereof.

35. The method of claim 32, wherein the transgene conveying improved resistance to temperature extremes is the CBF1 gene; the CBF4 gene; the DREB1A gene; or the E. coli MnSOD gene; or any combination thereof.

36. The method of claim 32, wherein the transgene conveying improved drought resistance is selected from the group consisting of the turgor responsive gene trg 31; the bacterial fructan genes; the δ-Pyrroline-5-Carboxylate Synthetase gene; the barley HVA1 gene; the Arabidopsis ERD1 gene; the mannitol-1-P dehydrogenase gene; a trehalose synthesis gene; NtC7 gene of tobacco; glutamine synthetase (GS) gene of rice; OsCDPL7 gene of rice; the DRO2 gene; the E. coli trehalose synthesis genes (TPSP/TPP); and the DREB2A gene; or any combination thereof.

37. The method of claim 32, wherein the transgene conveying improved drought resistance is the DRO2 gene, the DREB2A gene, a trehalose synthesis gene, or any combination thereof.

38. The method of claim 32, wherein the transgene conveying improved phenotypic characteristics is selected from the group consisting of the 2-oxidase gene; the OsGA20-ox2; the OsGA3-ox2; the BAS 1 gene; the rol (A, B, C) genes; the phyA gene; the crtO gene; the lycopene cyclase gene; OsMADS45 gene; and the OsMADS1 gene; or any combination thereof.

39. The method of claim 32, wherein the transgene conveying improved phenotypic characteristics is the 2-oxidase gene or the BAS1 gene, or a combination thereof.

40. The method of claim 32, wherein the transgene conveying improved disease resistance is selected from the group consisting of genes useful in combating southern lawn chinch bug; White grubs; Sod webworms; Armyworms; Cutworms; Fungal infections such as brown patch, gray leaf spot, Helminthosporium, Pythium, rust and downy mildew; viral infections such as SAD virus; or any combination thereof.

41. The method of claim 32, wherein the transgene conveying improved insect feeding resistance is selected from the group consisting of the Phaseolus vulgaris alpha amylase inhibitor; Arcelin 5A seed storage genes; the sweet potato trypsin inhibitor; the Bacillus thuringiensis cry1A and cry1B genes; the Nicotiana alata proteinase inhibitor gene; the Mir1 cysteine proteinase inhibitor; the chitinase gene; or any combination thereof.

42. The method of claim 32, wherein the transgenes conveying improved insect feeding resistance are the Bacillus thuringiensis cry1A and cry1B genes.

43. The method of claim 32, wherein the transgene conveying improved virus disease resistance is selected from the group consisting of viral coat protein genes; viral NSM genes; viral antisense RNA genes; nuclear inclusion genes; or any combination thereof.

44. The method of claim 32, wherein the transgene conveying improved microbial pathogen disease resistance is selected from the group consisting of beta 1,3-glucanase gene; the cecropin gene; the MeRIP gene of Mirabilis expansisn; the chitinase gene; antimicrobial peptide genes; or any combination thereof.

45. The method of claim 32, wherein the transgene conveying improved disease resistance is selected from the group consisting of the Rpg1 gene of barley; the NDR1 gene of Arabidopsis; various R genes from the superfamilies NB-LRR, eLRR and LRR-kinase; genes that encode transcription factors that regulate genes more directly involved in resistance to diseases organisms; or any combination thereof.

46. The method of claim 32, wherein the transgenes conveying improved disease resistance are various R genes from the superfamilies NB-LRR, eLRR and LRR-kinase or genes that encode transcription factors that regulate genes more directly involved in resistance to diseases organisms, or combinations thereof.

47. The method of claim 19, wherein the transgene is a heterologous promoter.

48. The method of claim 47, wherein the promoter is selected from the group consisting of the cauliflower mosaic virus 35S promoter; the figwort mosaic virus promoter; the maize ubiquitin promoter; the rice actin promoter; the nopaline synthase promoter; or any combination thereof.

49. The method of claim 47, wherein the promoter is selected from the group of promoters controlling the genes consisting of the rd29A gene of Arabidopsis; the Pcp1 gene of the ice plant; the blt4 gene of barley; the CPRD genes of cowpea; or any combination thereof.

50. The method of claim 47, wherein the promoter is selected from the group of promoters controlling the genes consisting of the POT9 gene of poplar; the TCH2 gene of Arabidopsis; the WAK14 gene of Arabidopsis; or any combination thereof.

51. The method of claim 19, wherein the callus tissue is transformed by microprojectile bombardment.

52. The method of claim 19, wherein the callus tissue is transformed by Agrobacterium-mediated DNA transfer.

53. The method of claim 19, wherein the callus tissue is transformed by DNA uptake following electroporation.

54. The method of claim 19, wherein the vector comprises a selectable marker and is selected from the group consisting of: pUC19 and pBIN19, derivatives of the same, or any combination thereof.

55. The method of claim 54, wherein the selectable marker is selected from the group consisting of an antibiotic, an enzyme, a herbicide resistance gene, or any combination thereof.

56. The method of claim 54, wherein the selectable marker is selected from the group consisting of CP4, nptII, bar, or any combination thereof.

57. The method of claim 25, wherein the selection medium is a hormone-free medium and the selective agent is glyphosate.
